(19) United States
(12) Patent Application Publication
(10) Pub. No.: US 2006/0030013 A1
(43) Pub. Date: Feb. 9, 2006

(54) Enzymatic synthesis of polyol acrylates

(75) Inventors: Wolfgang Paulus, Ober-Olm (DE); Bernhard Hauer, Fussgonheim (DE); Dietmar Haring, Schriesheim (DE); Frank Dietsche, Schriesheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/535,525

(22) PCT Filed: Nov. 21, 2003
(86) PCT No.: PCT/EP03/13106

§ 371 Date: Jul. 1, 2005

(30) Foreign Application Priority Data
Nov. 22, 2002 (DE) 102 54 642.8

(51) Int. Cl. C08F 118/02; C12P 7/62

(52) U.S. Cl. 435/135; 526/319

(57) ABSTRACT

The invention relates to a process for the enzymatic synthesis of polyol acrylates and also to a process for preparing polymeric polyol acrylates, to the polymers obtainable by this process, and to their use for preparing radiation-curable and thermally curable coating materials.

The invention relates to a process for the enzymatic synthesis of polyol acrylates and also to a process for preparing polymeric polyol acrylates, to the polymers obtainable by this process, and to their use for preparing radiation-curable and/or thermally curable coating materials.

### PRIOR ART

The polyol acrylates are obtainable in a variety of ways. Polyol acrylates are chemically synthesized by direct esterification or transesterification of acrylic acid or acrylic esters with polyols, which takes place at temperatures above 100° C. under acid catalysis. Owing to the high temperatures it is necessary to add large amounts of polymerization inhibitors. The product mixtures which result are complex and often dark. Impurities either are removed from the product solution by complicated alkaline washes, along with the superstoichiometric acrylic acid, or remain in the product. The washing procedure is protracted and expensive, since partly esterified products in particular are slow to extract and result in poor yields owing to the relatively high hydrophilicity of the products. The composition in the case of higher polyols is shifted toward the more highly acrylated products, owing to the high excess of acrylic acid. Such products are undesirable in thermosetting systems, since they dissolve out of the film, diffuse to the surface, and, in a way which is very negative for their use, may give rise, as a softening component in films which cure by means of heat alone, to tacky surfaces (see V1).

An alternative route to polyol acrylates is by ring-opening addition reaction of oxiranes with acrylic acid. These products are generally characterized by a broad spectrum of byproducts, since the starting materials result from reactions of alcohols with epichlorohydrin; that is, the chlorine content is very high owing to the nonregioselective reaction.

As far as biocatalytic synthesis is concerned, essentially two different pathways have been taken to date. The first preparation pathway involves the use of activated acrylic acid derivatives. Known in particular are biocatalytic syntheses of this kind with vinyl (meth)acrylate (e.g., Derango et al., Biotechnol Lett. 1994, 16, 241-246); butanediol monooxime esters of (meth)acrylic acid (Athawale and Manjrekar, J. Mol. Cat. B Enzym. 2000, 10, 551-554) or trifluoroethyl(meth)acrylate (Potier et al., Tetrahedron Lett. 2000, 41, 3597-3600). Because of their high production costs, however, activated acrylic acid derivatives of this kind are of no interest for an economic synthesis of polyol acrylates.

Alcohol acrylates can also be prepared biocatalytically by enzymatic esterification or transesterification of acrylic acid or alkyl acrylates with different alcohols.

For example, JP-A-59220196 describes the esterification of acrylic acid with diols in aqueous phosphate buffer with the aid of a crude enzyme extract from Alcaligenes sp. and unsaturated fatty alcohols can be transesterified enzymatically with methyl or ethyl acrylate (Warwel et al., Biotechnol Lett. 1996, 10, 283-286). Nurok et al. (J. Mol. Cat. B Enzym. 1999, 7, 273-282) describe the lipase-catalyzed transesterification of 2-ethylhexanol with methyl acrylate. The enzymatic transesterification of cyclic and open-chain alkanediols with ethyl acrylate is accomplished using a lipase from Chromobacterium viscosum (Hajjar et al., Biotechnol. Lett. 1990, 12, 825-830). In U.S. Pat. No. 5,240,835 (Genencor International Inc., 1989) the transesterification of alkyl acrylates with alcohols with catalysis by a biocatalyst from Corynebacteium oxydans is described. By way of example, in that document, a 96-fold molar excess of ethyl acrylate is reacted with 2,2-dimethyl-1,3-propanediol. A yield of only 21% is obtained after 3 days at 30° C. Tor et al. (Enzym. Microb. Technol. 1990, 12, 299-304) esterified ethylene glycol, diethylene glycol, triethylene glycol, propylene glycol, 1,4-butanediol, and glycerol with methyl or ethyl(meth)acrylate. The reactions were catalyzed by pig liver esterase (PLE) which had been treated with glutaraldehyde and polyacrylamide-hydrazide. This special pretreatment of the enzyme was necessary to stabilize it with respect to the aqueous substrate solution. Glycerol was esterified at a substrate concentration of 20 mM and the solution contained 30% by volume of a 50 mM phosphate buffer (cf. also IL 090820, 1989). EP-A-999 229 (Goldschmidt AG, 1999) describes the lipase-catalyzed transesterification of (meth)acrylic acid or alkyl(meth)acrylates with polyoxyalkylenes (e.g., polyethylene glycol). Suitable polyoxyalkylenes contain 4-200, preferably 8-120, oxyalkylene units.

A process for the enzymatic synthesis of sugar acrylates is described in the older DE-A-101 56 352.3.

The biocatalytic preparation of acrylates of polyhydric (3 or more hydroxyl groups) alcohols, especially those which are aliphatic and cyclic or noncyclic, however, has not been hitherto described. In particular, the enzymatic preparation of aliphatic polyols with low levels of acrylicization, i.e., incompletely acrylated polyols, is unknown from the prior art.

These compounds are of particular interest for use in dual-cure systems. It will be desirable to combine the very positive mechanical properties of radiation-curable coating materials with the additional option of a thermal cure owing to incomplete curing in shadow regions when coating three-dimensional objects. The aim is for a highly scratch-resistant, odorless, and tack-free surface on different substrates. This aim is difficult to achieve using current products, since the conventional esterification produces very high fractions of completely acrylated or completely unacrylated products, which remain extractable following curing either by means of heat alone or by means of radiation alone.

### SHORT DESCRIPTION OF THE INVENTION

It is an object of the present invention to develop a process for preparing acrylates of polyhydric aliphatic alcohols. The synthesis ought in particular to be implementable with a good yield of products with low degrees of acrylicization, such as polyol monoacrylate and polyol diacrylate, for example, but also to lead to completely esterified products. In particular there should be no aqueous workup/extraction of the products.

We have found that this object is achieved, surprisingly, by a skillful choice of the process conditions, in particular by working in an organic medium.

### DETAILED DESCRIPTION OF THE INVENTION

The invention firstly provides a process for the enzymatic synthesis of polyol acrylates, in which an aliphatic polyol is reacted with an acrylic acid compound or an alkyl ester thereof in bulk or in a liquid reaction medium comprising an organic solvent, in the presence of an enzyme which transfers acrylate groups, and after the end of the reaction the polyol acrylate(s) formed is(are) isolated if desired from the reaction mixture.

An “aliphatic polyol acrylate” for the purposes of the invention is singly or multiply acrylated.

When the process of the invention is implemented the reaction product obtained preferably contains, based on the overall amount of acrylated polyols, polyols with low degrees of acrylicization in a molar fraction of about 20 to 100 mol %, more preferably 40 to 99 mol %, in particular 50 to 95 mol % or 60 to 90 mol %.

In a “polyol with a low degree of acrylicization” for the purposes of the invention the ratio B/A of acrylicizable hydroxyl groups prior to the reaction (A) and acrylicizable hydroxyl groups remaining after the reaction (B) is <1, such as, for example, 0.1 to 0.9 or 0.2 to 0.66.

The reaction product of the invention preferably constitutes, moreover, a product mixture in which the sum of fully acrylated and completely unacrylated polyols after the reaction amounts to less than 20% by weight, in particular less than 10% by weight, based in each case on the total weight of the reaction mixture minus the weight of any solvent and/or low molecular mass additives present.

In accordance with one specific embodiment of the invention the reaction product of the invention can be obtained by adding completely acrylated compounds to the reaction mixture and allowing the esterification reaction to equilibrate.

The conversion achieved in accordance with the invention (the molar fraction of polyol acrylate esters which carry at least one ester group) lies in accordance with the invention at not less than 20 mol %, such as, for example, 20 to 100 mol %, 40 to 99 mol %, 50 to 95 mol % or 75 to 95 mol %, based in each case on the moles of polyol employed.

The liquid organic reaction medium may have an initial water content of up to about 10% by volume, is preferably substantially anhydrous. The reaction can take place in bulk or else, if advantageous, after a suitable organic solvent has been added.

Organic solvents used include preferably those selected from monools, such as C₁-C₆ alkanols, such as methanol, ethanol, 1- or 2-propanol, tert-butanol, and tert-amyl alcohol, for example, pyridine, poly-C₁-C₄ alkylene glycol di-C₁-C₄ alkyl ethers, especially polyethylene glycol di-C₁-C₄ alkyl ethers, such as dimethoxyethane, diethylene glycol dimethyl ether, polyethylene glycol dimethyl ether 500, C₁-C₄ alkylene carbonates, especially propylene carbonate, C₁-C₆ alkyl acetates, in particular tert-butyl acetates, MTBE, acetone, 1,4-dioxane, 1,3-dioxolane, THF, dimethoxymethane, dimethoxyethane, cyclohexane, methylcyclohexane, toluene, hexane, and single-phase or multiphase mixtures thereof.

In the process of the invention acrylic acid compound and polyol are used generally in a molar ratio of about 100:1 to 1:1, such as, for example, in the range from 30:1 to 3:1 or 10:1 to 5:1.

The initial polyol concentration lies, for example, in the range of about 0.1 to 20 mol/l, in particular 0.15 to 10 mol/l.

The polyol is preferably selected from straight-chain, branched, and carbocyclic, saturated and unsaturated hydrocarbon compounds having at least 3 carbon atoms and at least 3 (esterifiable) hydroxyl groups in optically pure form or as a stereoisomer mixture. Unsaturated hydrocarbon compounds may have 1 or more, preferably 1, 2 or 3 C—C double bonds. Mixtures of such polyols are likewise employable.

The polyol is in particular a straight-chain or branched saturated hydrocarbon having 3 to 30 carbon atoms and 3 to 10 hydroxyl groups.

Preferred examples of polyols which can be used include the following: glycerol, di-, tri-, and polyglycerols, low molecular mass, partly or fully hydrolyzed polyvinyl acetate, 1,2,4-butanetriol, trimethylolmethane, trimethylolethane, trimethylolpropane, trimethylolbutane, 2,2,4-trimethyl-1,3-pentanediol, pentaerythritol, ditrimethylolpropane, dipentaerythritol, tripentaerythritol, D-, L-, and mesoerythritol, D- and L-arabitol, adonitol, xylitol, sorbitol, mannitol, dulcitol and inositols, and also mixtures and derivatives thereof. By “derivatives” are meant in particular C₁-C₆ alkyl ethers, such as methyl ethers, for example; C₁-C₄ alkylene ethers, such as ethylene or propylene glycol ethers, for example, or esters of saturated or unsaturated C₁-C₂₀ carboxylic acids. Inventively employed polyols and their derivatives contain in particular no polyoxyalkylene groups having four or more oxyalkylene units, such as the polyoxyalkylenes used in accordance with EP-A-0 999 229, for example. Preferred polyols or derivatives thereof contain no polyoxyalkylene units.

The inventively employed “acrylic acid compound” is preferably selected from acrylic acid, its anhydrides, lower-alkyl-substituted—i.e., C₁-C₆ alkyl-substituted—acrylic acid, the C₁-C₂₀ alkyl esters thereof or ethylene glycol diacrylates; and mixtures of these compounds. Preferred C₁-C₆ alkyl groups are, in particular, methyl or ethyl groups. Examples of preferred C₁-C₂₀ alkyl groups include methyl, ethyl, i- or n-propyl, n-, i-, sec- or tert-butyl, n- or i-pentyl; furthermore, n-hexyl, n-heptyl, n-octyl, n-nonyl, n-decyl, n-undecyl, n-tridecyl, n-tetradecyl, n-pentadecyl and n-hexadecyl, and n-octadecyl, and also the singly or multiply branched analogs thereof. Preference is given to using (meth)acrylic acid or (meth)acrylic acid derivatives.

Suitable derivatives of above acrylic acid compounds, such as acrylic and methacrylic acid, for example, are esters with saturated and unsaturated, cyclic or open-chain C₁-C₁₀ monoalcohols, particularly the methyl, ethyl, butyl, and 2-ethylhexyl esters thereof. The C₁-C₁₀ monoalcohols according to the invention include preferably C₁-C₆ alkyl groups as defined above or their longer-chain, optionally branched, homologs having up to 10 carbon atoms or C₄-C₆ cycloalkyl groups, such as cyclopropyl, cyclopentyl or cyclohexyl, which may where appropriate have been substituted by one or more alkyl groups having 1 to 3 carbon atoms.

Unless specified otherwise, C₁-C₆ alkyl according to the invention stands for methyl, ethyl, n- or i-propyl, n-, sec- or tert-butyl; n- or tert-amyl, and also straight-chain or branched hexyl. C₃-C₆ alkyl stands in particular for n- or i-propyl, n-, sec- or tert-butyl, n- or tert-amyl, and also straight-chain or branched hexyl. C₁-C₄ alkylene stands preferably for methylene, ethylene, propylene or 1- or 2-butylene.

The enzymes used in accordance with the invention are selected from hydrolases, preferably esterases (E.C. 3.1.-.-), such as in particular lipases (E.C. 3.1.1.3), glycosylases (E.C. 3.2.-.-) and proteases (E.C. 3.4.-.-) in free or immobilized form. Particularly suitable are Novozyme 435 (lipase from Candida antarctica B) or lipase from Aspergillus sp., Burkholderia sp., Candida sp., Pseudomonas sp., or porcine pancreas. The enzyme content of the reaction medium lies in particular in the range from about 0.1 to 10% by weight, based on the polyol used. In the reaction according to the invention the enzymes can be used in pure form or supported (immobilized).

The process of the invention is preferably conducted so that the reaction temperature is in the range from 0 to about 100° C., in particular in the range from 20 to 80° C. The reaction time is generally in the range from about 3 to 72 hours.

Any alcohol obtained during the transesterification (generally a monohydric alcohol, such as methanol or ethanol) or the water of reaction produced during the esterification may be removed, if necessary, from the reaction equilibrium in an appropriate fashion, continuously or in steps. Suitable for this purpose are preferably molecular sieves (pore size, for example, in the region of about 3-10 Angstroms), or separation by distillation, by suitable semipermeable membranes or by pervaporation.

To mix the reaction batch it is possible to use any desired methods. Special stirring equipment is not needed. The reaction medium may be single-phase or multiphase and the reactants are introduced in solution, suspension or emulsion therein, together where appropriate with the molecular sieve. At the start of the reaction the medium can be admixed with the enzyme preparation. The temperature is set during the reaction at the desired level.

Alternatively, the reaction can be carried out such that the enzyme is charged in immobilized form to a fixed bed reactor and the reaction batch is pumped over the immobilized enzyme, where appropriate in circulation. Water of reaction and/or alcohol of reaction can likewise be removed continuously or in steps from the reaction mixture.

The process of the invention can be carried out batchwise, semicontinuously or continuously in conventional bioreactors. Suitable regimes and bioreactors are familiar to the skilled worker and are described, for example, in Römpp Chemie Lexikon, 9th edition, Thieme Verlag, entry header “Bioreactor” or Ullmann's Encyclopedia of Industrial Chemistry, 5th edition, volume B4, page 381 ff., hereby incorporated by reference. The operation of the reactor and the process regime can be adapted by the skilled worker to the particular requirements of the desired esterification reaction.

After the end of the reaction the desired polyol acrylate can be isolated from the reaction mixture, such as by chromatographic purification, and then used to prepare the desired polymers or copolymers.

The invention further provides a process for preparing polymeric polyol acrylates wherein at least one polyol acrylate is prepared as described above separated if desired from the reaction mixture, and polymerized if desired together with further comonomers.

Suitable further comonomers are the following: other inventively prepared polyol acrylates of the inventive type or polymerizable monomers such as (meth)acrylic acid, maleic acid, itaconic acid, the alkali metal salts or ammonium salts thereof and the esters thereof, O-vinyl esters of C₁-C₂₅ carboxylic acids, N-vinylamides of C₁-C₂₅ carboxylic acids, N-vinylpyrrolidone, N-vinylcaprolactam, N-vinyloxazolidone, N-vinylimidazole, quaternized N-vinylimidazole, (meth)acrylamide, (meth)acrylonitrile, ethylene, propylene, butylene, butadiene, styrene. Examples of suitable C₁-C₂₅ carboxylic acids are saturated acids, such as formic, acetic, propionic, and n- and i-butyric acid, n- and i-valeric acid, caproic acid, enanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotinic acid, and melissic acid.

The preparation of such polymers takes place for example in analogy to the processes described in general in Ullmann's Encyclopedia of Industrial Chemistry, Sixth Edition, 2000, Electronic Release, entry heading: Polymerisation Process. The (co)polymerization preferably takes place as a free-radical addition polymerization in the form of solution, suspension, precipitation or emulsion polymerization or by polymerization in bulk, i.e., without solvent.

The invention further provides a process for preparing polymeric polyol acrylates wherein at least one polyol acrylate is prepared as described above and the incompletely esterified polyol acrylate is separated if desired from the reaction mixture and polymerized if desired together with further comonomers.

Examples of suitable comonomers include the following: other inventively prepared polyol acrylates of the inventive type or polymerizable monomers such as ethylene oxide and propylene oxide, for example.

The preparation of such polymers takes place with metallic catalysis without alkaline ester cleavage, as is the case with, for example, U.S. Pat. No. 6,359,101, DE 198 17 676, DE 199 13 260, U.S. Pat. No. 6,429,342; U.S. Pat. No. 6,077,979 and U.S. Pat. No. 5,545,601.

The invention further provides for the use of the polyol acrylates of the invention for preparing coating materials and especially radiation-curable compositions, such as radiation-curable coating materials in particular. This is done using polyol acrylates, such as glyceryl acrylates, trimethylolpropane triacrylates or pentaerythritol acrylates, for example, in the form of their mono-, di- or polyacrylates (and/or mixtures thereof, as homopolymers or copolymers for radiation-curing coating materials in, for example, dual cure systems. Such systems are described in, for example, WO-A-98/00456, which is expressly incorporated by reference.

Besides the polyol acrylates (A) obtainable by the process of the invention a radiation-curable composition of the invention may comprise the following components:
(B) at least one polymerizable compound other than (A), containing two or more copolymerizable ethylenically unsaturated groups,
(C) if desired, reactive diluents,
(D) if desired, photoinitiator, and
(E) if desired, further typical coatings additives.

Suitable compounds (B) include radiation-curable, free-radically polymerizable compounds containing two or more copolymerizable ethylenically unsaturated groups.

Compounds (B) are preferably vinyl ether or (meth)acrylate compounds, more preferably in each case the acrylate compounds, i.e., the derivatives of acrylic acid. Preferred vinyl ether and (meth)acrylate compounds (B) contain up to 20, more preferably up to 10, and very preferably up to 6, such as 2, 3, 4 or 5, copolymerizable ethylenically unsaturated double bonds.

Particularly preferred compounds (B) are those having an ethylenically unsaturated double bond content of 0.1-0.7 mol/100 g, very preferably 0.2-0.6 mol/100 g.

The number-average molecular weight Mₙ of the compounds (B), unless indicated otherwise, is preferably below 15 000, more preferably 300-12 000, very preferably 400 to 5000, and in particular 500-3000 g/mol (as determined by gel permeation chromatography using polystyrene as standard and tetrahydrofuran as eluent).

Examples of compounds (B) include the following: (meth)acrylate compounds, such as (meth)acrylic esters and especially acrylic esters; and also vinyl ethers of monohydric or polyhydric alcohols, particularly those which other than the hydroxyl groups contain no functional groups or, if any at all, then ether groups. Examples of monohydric alcohols are particularly methanol, ethanol, and n- and i-propanol. Examples of such polyhydric alcohols are difunctional alcohols, such as ethylene glycol, propylene glycol, and their counterparts with higher degrees of condensation, such as diethylene glycol, triethylene glycol, dipropylene glycol, tripropylene glycol, etc.; 1,2-, 1,3- or 1,4-butanediol, 1,5-pentanediol, 1,6-hexanediol, 3-methyl-1,5-pentanediol, neopentyl glycol, alkoxylated phenolic compounds, such as ethoxylated and/or propoxylated bisphenols, 1,2-, 1,3- or 1,4-cyclohexanedimethanol, trifunctional and higher polyfunctional alcohols, such as glycerol, trimethylolpropane, butanetriol, trimethylolethane, pentaerythritol, ditrimethylolpropane, dipentaerythritol, sorbitol, mannitol, and the corresponding alkoxylated, especially ethoxylated and/or propoxylated, alcohols.

The alkoxylation products are obtainable conventionally by reacting the above alcohols with alkylene oxides, especially ethylene oxide or propylene oxide. The degree of alkoxylation per hydroxyl group is preferably from 0 to 10; that is, 1 mol of hydroxyl group can have been alkoxylated with up to 10 mol of alkylene oxides.

Further suitable (meth)acrylate compounds include polyester (meth)acrylates, which are the (meth)acrylic esters or vinyl ethers of polyesterols, and also urethane, epoxy or melamine (meth)acrylates.

Urethane (meth)acrylates, for example, are obtainable by reacting polyisocyanates with hydroxyalkyl(meth)acrylates and, if desired, chain extenders such as diols, polyols, diamines, polyamines or dithiols or polythiols.

The urethane (meth)acrylates preferably have a number-average molar weight Mₙ of from 500 to 20 000, in particular from 750 to 10 000, more preferably from 750 to 3000 g/mol (as determined by gel permeation chromatography using polystyrene as standard).

The urethane (meth)acrylates preferably contain from 1 to 5, more preferably from 2 to 4, mol of (meth)acrylic groups per 1000 g of of urethane (meth)acrylate.

Epoxy(meth)acrylates are obtainable by reacting epoxides with (meth)acrylic acid. Examples of suitable epoxides include epoxidized olefins or glycidyl ethers, e.g., bisphenol A diglycidyl ether or aliphatic glycidyl ethers, such as butanediol diglycidyl ethers.

Melamine(meth)acrylates are obtainable by reacting melamine with (meth)acrylic acid or the esters thereof.

The epoxy(meth)acrylates and melamine(meth)acrylates preferably have a number-average molar weight Mₙ of from 500 to 20 000, more preferably from 750 to 10 000 g/mol and very preferably from 750 to 3000 g/mol; the amount of (meth)acrylic groups is preferably from 1 to 5, more preferably from 2 to 4, per 1000 g of of epoxy (meth)acrylate or melamine(meth)acrylate (as determined by gel permeation chromatography using polystyrene as standard and tetrahydrofuran as eluent).

Also suitable are carbonate(meth)acrylates containing on average preferably from 1 to 5, in particular from 2 to 4, more preferably from 2 to 3 (meth)acrylic acid groups and very preferably 2 (meth)acrylic groups.

The number-average molecular weight Mₙ of the carbonate(meth)acrylates is preferably less than 3000 g/mol, more preferably less than 1500 g/mol, very preferably less than 800 g/mol (as determined by gel permeation chromatography using polystyrene as standard with tetrahydrofuran as solvent).

The carbonate(meth)acrylates are obtainable in simple fashion by transesterifying carbonic esters with polyhydric, preferably dihydric, alcohols (diols, e.g., hexanediol) and subsequently esterifying the free OH groups with (meth)acrylic acid or else by transesterification with (meth)acrylic esters, as described in, for example, EP-A 92 269. They are also obtainable by reacting phosgene, urea derivatives with polyhydric, e.g., dihydric, alcohols.

Suitable reactive diluents (compounds (C)) include radiation-curable, free-radically or cationically polymerizable compounds having only one ethylenically unsaturated copolymerizable group.

Examples that may be mentioned include C₁-C₂₀ alkyl (meth)acrylates, vinylaromatics having up to 20 carbon atoms, vinyl esters of carboxylic acids containing up to 20 carbon atoms, ethylenically unsaturated nitriles, vinyl ethers of alcohols containing 1 to 10 carbon atoms, α,β-unsaturated carboxylic acids and their anhydrides, and aliphatic hydrocarbons having 2 to 8 carbon atoms and 1 or 2 double bonds.

Preferred (meth)acrylic acid alkyl esters are those with a C₁-C₁₀ alkyl radical, such as methyl methacrylate, methyl acrylate, n-butyl acrylate, ethyl acrylate and 2-ethylhexyl acrylate.

Also suitable in particular are mixtures of the (meth)acrylic acid alkyl esters.

Vinyl esters of carboxylic acids having 1 to 20 carbon atoms are, for example, vinyl laurate, vinyl stearate, vinyl propionate, and vinyl acetate.

α,β-Unsaturated carboxylic acids and their anhydrides may be, for example, acrylic acid, methacrylic acid, fumaric acid, crotonic acid, itaconic acid, maleic acid or maleic anhydride, preferably acrylic acid.

Suitable vinylaromatic compounds include for example vinyltoluene, α-butylstyrene, 4-n-butylstyrene, 4-n-decylstyrene, and, preferably, styrene.

Examples of nitriles are acrylonitrile and methacrylonitrile.

Examples of suitable vinyl ethers are vinyl methyl ether, vinyl isobutyl ether, vinyl hexyl ether, and vinyl octyl ether.

Nonaromatic hydrocarbons having 2 to 8 carbon atoms and one or two olefinic double bonds that may be mentioned include butadiene, isoprene, and also ethylene, propylene, and isobutylene.

It is additionally possible to use N-vinylformamide, N-vinylpyrrolidone, and N-vinylcaprolactam.

As photoinitiators (D) it is possible to use those which are known to the skilled worker, examples being those specified in “Advances in Polymer Science”, Volume 14, Springer Berlin 1974 or in K. K. Dietliker, Chemistry and Technology of UV- and EB-Formulation for Coatings, Inks and Paints, Volume 3; Photoinitiators for Free Radical and Cationic Polymerization, P. K. T. Oldring (Ed.), SITA Technology Ltd, London.

Examples that may be considered include mono- or bisacylphosphine oxides Irgacure 819 (bis(2,4,6-trimethylbenzoyl)phenylphosphine oxide), as described in, for example, EP-A 7 508, EP-A 57 474, DE-A 196 18 720, EP-A 495 751 or EP-A 615 980, such as 2,4,6-trimethylbenzoyl-diphenylphosphine oxide (Lucirin™ TPO), ethyl 2,4,6-trimethylbenzoylphenylphosphinate, benzophenones, hydroxyacetophenones, phenylglyoxylic acid and its derivatives, or mixtures of these photoinitiators. Examples include benzophenone, acetophenone, acetonaphthoquinone, methyl ethyl ketone, valerophenone, hexanophenone, α-phenylbutyrophenone, p-morpholino propiophenone, dibenzosuberone, 4-morpholinobenzophenone, 4-morpholinodeoxybenzoin, p-diacetylbenzene, 4-aminobenzophenone, 4′-methoxyacetophenone, β-methylanthraquinone, tert-butylanthraquinone, anthraquinoncarboxylic esters, benzaldehyde, α-tetralone, 9-acetyl phenanthrene, 2-acetylphenanthrene, 10-thioxanthenone, 3-acetylphenanthrene, 3-acetylindole, 9-fluorenone, 1-indanone, 1,3,4-triacetylbenzene, thioxanthen-9-one, xanthen-9-one, 2,4-dimethylthioxanthone, 2,4-diethylthioxanthone, 2,4-di-iso-propylthioxanthone, 2,4-dichloro thioxanthone, benzoin, benzoin iso-butyl ether, chloroxanthenone, benzoin tetrahydropyranyl ether, benzoin methyl ether, benzoin ethyl ether, benzoin butyl ether, benzoin iso-propyl ether, 7H-benzoin methyl ether, benz[de]anthracen-7-one, 1-naphthaldehyde, 4,4′-bis(dimethylamino)benzophenone, 4-phenylbenzophenone, 4-chlorobenzophenone, Michler's ketone, 1-acetonaphthone, 2-acetonaphthone, 1-benzoylcyclohexan-1-ol, 2-hydroxy-2,2-dimethyl acetophenone, 2,2-dimethoxy-2-phenylacetophenone, 2,2-diethoxy-2-phenylacetophenone, 1,1-dichloroacetophenone, 1-hydroxyacetophenone, acetophenone dimethyl ketal, o-methoxy benzophenone, triphenylphosphine, tri-o-tolylphosphine, benz[a]anthracene-7,12-dione, 2,2-diethoxyacetophenone, benzil ketals, such as benzil dimethyl ketal, 2-methyl-1-[4-(methylthio)phenyl]-2-morpholinopropan-1-one, anthraquinones such as 2-methyl anthraquinone, 2-ethylanthraquinone, 2-tert-butylanthraquinone, 1-chloroanthraquinone, 2-amylanthraquinone, and 2,3-butanedione.

Also suitable are nonyellowing or low-yellowing photoinitiators of the phenylglyoxalic ester type, as described in DE-A 198 26 712, DE-A 199 13 353 or WO 98/33761.

Among the specified photoinitiators preference is given to phosphine oxides, α-hydroxy ketones, and benzophenones.

In particular it is also possible to use mixtures of different photoinitiators.

The photoinitiators can be used alone or in combination with a photopolymerization promoter, of the benzoic acid, amine or similar type, for example.

As further typical coatings additives (E) it is possible, for example, to use antioxidants, oxidation inhibitors, stabilizers, activators (accelerators), fillers, pigments, dyes, devolatilizers, luster agents, antistats, flame retardants, thickeners, thixotropic agents, leveling assistants, binders, antifoams, fragrances, surface-active agents, viscosity modifiers, plasticizers, plastifying agents, tackifying resins (tackifiers), chelating agents or compatibilizers.

As accelerators for the thermal aftercure it is possible to use, for example, tin octoate, zinc octoate, dibutyltin dilaurate or diaza[2.2.2]bicyclooctane.

It is additionally possible to add one or more photochemically and/or thermally activatable initiators, e.g., potassium peroxodisulfate, dibenzoyl peroxide, cyclohexanone peroxide, di-tert-butyl peroxide, azobis-iso-butyronitrile, cyclohexylsulfonyl acetyl peroxide, di-iso-propyl percarbonate, tert-butyl peroctoate or benzpinacol, and also, for example, thermally activatable initiators having a half-life at 80° C. of more than 100 hours, such as di-t-butyl peroxide, cumene hydroperoxide, dicumyl peroxide, t-butyl perbenzoate, silylated pinacols, which are available commercially, for example, under the trade name ADDID 600, from Wacker, or hydroxyl-containing amine N-oxides, such as 2,2,6,6-tetramethylpiperidine-N-oxyl, 4-hydroxy-2,2,6,6-tetramethylpiperidine-N-oxyl, etc. Further examples of suitable initiators are described in “Polymer Handbook”, 2nd edition, Wiley & Sons, New York.

Suitable thickeners, as well as free-radically (co)polymerized (co)polymers include customary organic and inorganic thickeners such as hydroxymethylcellulose or bentonites.

Examples of chelate formers which can be used include ethylenediamineacetic acid and its salts and also β-diketones.

Suitable fillers include silicates, such as the silicates obtainable by hydrolyzing silicon tetrachloride, such as Aerosil® from Degussa, siliceous earth, talc, aluminum silicates, magnesium silicates, calcium carbonates, etc.

Suitable stabilizers include typical UV absorbers such as oxanilides, triazines, and benzotriazole (the latter obtainable as Tinuvin® grades from Ciba Spezialitatenchemie), and benzophenones. These can be used alone or together with suitable free-radical scavengers, examples being sterically hindered amines such as 2,2,6,6-tetramethylpiperidine, 2,6-di-tert-butylpiperidine or derivatives thereof, e.g., bis-(2,2,6,6-tetramethyl-4-piperidyl)sebacate. Stabilizers are used commonly in amounts of from 0.1 to 5.0% by weight, based on the solid components present in the formulation.

Examples of stabilizers suitable additionally include N-oxyls, such as 4-hydroxy-2,2,6,6-tetramethylpiperidine-N-oxyl, 4-oxo-2,2,6,6-tetramethylpiperidine-N-oxyl, 4-acetoxy-2,2,6,6-tetramethylpiperidine-N-oxyl, 2,2,6,6-tetramethylpiperidine-N-oxyl, 4,4′,4″-tris(2,2,6,6-tetramethylpiperidine-N-oxyl)phosphite or 3-oxo-2,2,5,5-tetramethylpyrrolidine-N-oxyl, phenols and naphthols, such as p-aminophenol, p-nitrosophenol, 2-tert-butylphenol, 4-tert-butylphenol, 2,4-di-tert-butylphenol, 2-methyl-4-tert-butylphenol, 4-methyl-2,6-tert-butylphenol (2,6-tert-butyl-p-cresol) or 4-tert-butyl-2,6-dimethylphenol, quinones, such as hydroquinone or hydroquinone monomethyl ether, aromatic amines, such as N,N-diphenylamine, N-nitrosodiphenylamine, phenylenediamines, such as N,N′-dialkyl-para-phenylenediamine, the alkyl radicals being identical or different, linear or branched, and independently of 1 to 4 carbon atoms, hydroxylamines, such as N,N-diethylhydroxylamine, urea derivatives, such as urea orthiourea, phosphorous compounds, such as triphenylphosphine, triphenyl phosphite ortriethyl phosphite, or sulfur compounds, such as diphenyl sulfide or phenothiazine.

Typical compositions of radiation-curable compositions are for example
(A) 20-100% by weight, preferably 40-90, more preferably 50-90, and especially 60-80% by weight,
(B) 0-60% by weight, preferably 5-50, more preferably 10-40, and especially 10-30% by weight,
(C) 0-50% by weight, preferably 5-40, more preferably 6-30, and especially 10-30% by weight,
(D) 0-20% by weight, preferably 0,5-15, more preferably 1-10, and especially 2-5% by weight, and
(E) 0-50% by weight, preferably 2-40, more preferably 3-30, and especially 5-20% by weight,
   with the proviso that (A), (B), (C), (D) and (E) together make 100% by weight.

The coating of substrates with coating compositions of the invention takes place by customary methods which are known to the skilled worker, in the course of which at least one coating composition is applied in the desired thickness to the substrate to be coated and any volatile constituents present in the coating composition are removed, where appropriate with heating. This operation may if desired be repeated one or more times. Application to the substrate may take place in a known way, for example, by spraying, troweling, knifecoating, brushing, rolling, roller coating, casting, laminating, backmolding or coextrusion. The coating thickness is generally in a range from about 3 to 1000 g/m² and preferably from 10 to 200 g/m².

Further disclosed is a method of coating substrates wherein the coating composition is applied to the substrate and dried where appropriate, cured with electron beams or UV light under an oxygen-containing atmosphere or, preferably, under inert gas, and treated thermally where appropriate at temperatures up to the level of the drying temperature and thereafter at temperatures up to 160° C., preferably between 60 and 160° C.

The method of coating substrates can also be conducted such that after the coating composition has been applied it is first treated thermally at temperatures up to 160° C., preferably between 60 and 160° C., and then cured with electron beams or UV light under oxygen or, preferably, under inert gas.

The curing of the films formed on the substrate may if desired take place exclusively by thermal means. Generally, however, the coatings are cured both by exposure to high-energy radiation and thermally.

Curing may also be effected, in addition to or instead of the thermal cure, by NIR radiation, NIR radiation referring here to electromagnetic radiation in the wavelength range from 760 nm to 2.5×10⁻⁷⁻ m, preferably from 900 to 1500 nm.

If desired, if two or more coats of the coating composition are applied one over another, each coating operation may be followed by a thermal, NIR and/or radiation cure.

Examples of suitable radiation sources for the radiation cure include low-pressure, medium-pressure, and high-pressure mercury lamps and also fluorescent tubes, pulsed emitters, metal halide lamps, electronic flash devices, which allow a radiation cure without photoinitiator, or excimer emitters. The radiation cure takes place by exposure to high-energy radiation, i.e., UV radiation or daylight, preferably light in the wavelength range λ=200 to 700 nm, more preferably λ=200 to 500 nm, and very preferably λ=250 to 400 nm, or by exposure to high-energy electrons (electron beams; 150 to 300 keV). Examples of radiation sources used include high-pressure mercury vapor lamps, lasers, pulsed lamps (flash lights), halogen lamps or excimer emitters. The radiation dose normally sufficient for crosslinking in the case of UV curing is in the range from 80 to 3000 mJ/cm².

Naturally it is also possible to use two or more radiation sources for curing, e.g., two to four. These sources may also each emit in different wavelength ranges:

Irradiation can where appropriate be carried out in the absence of oxygen, e.g., under an inert gas atmosphere. Suitable inert gases include preferably nitrogen, noble gases, carbon dioxide, or combustion gases. Irradiation can also take place with the coating composition covered with transparent media. Examples of transparent media include polymer films, glass or liquids, e.g., water. Particular preference is given to irradiation in the manner described in DE-A1 199 57 900.

The invention further provides a method of coating substrates wherein
i) a substrate is coated with a coating composition as described above,
ii) volatile constituents of the coating material are removed to form a film under conditions in which the photoinitiator (C) substantially does not yet form any free radicals,
iii) if desired, the film formed in step ii) is exposed to high-energy radiation, in which case the film is precured, and subsequently, if desired, the article coated with the precured film is machined or the surface of the precured film is contacted with another substrate, and
iv) the curing of the film is completed thermally or with NIR radiation.

Steps iv) and iii) here may also be carried out in the opposite order, i.e., the film can be cured first thermally or by NIR radiation and then with high-energy radiation.

Further provided with the present invention are substrates coated with a coating composition of the invention.

The invention is now illustrated with reference to the following examples.

General Details:

A) Gas Chromatography:

The reaction products of glycerol and trimethylolpropane with the acrylates were separated by gas chromatography on a capillary column CP-Sil 19 (14% cyanopropylphenyl, 86% dimethyl-polysiloxanes) from Varian. For the GC analysis of the reaction products of sorbitol and erythritol with acrylates, 50 μl of reaction solution were treated with 950 μl of Sylon HTP (from Supelco) at 20° C. for 10 minutes and then analyzed on a capillary column CP-Sil 5 (100% dimethylpolysiloxanes, from Varian).

B) Determination of “Total Extractables”:

The fraction of total extractables in thermally cured coating materials is determined by acetone extraction of tablets of thermally cured coating material.

a) Preparation of the Coating Material Tablets and Testing:

The coating materials under test are prepared freshly (without photoinitiator) and weighed out (5 g). The coating material tablets are cured in a drying cabinet at 60° C. for 24 h. After curing, the films are halved. Each half is weighed (analytical balance, one beaker for the extraction and one beaker without acetone for comparison). One beaker (Ac) is filled with 100 g of of acetone. Both beakers are closed with lids and stored at 23° C./55% relative humidity for 24 h.

Following storage, the acetone is poured from the Ac beakers (through a nylon sieve, so as to retain any tablet fragments). All beakers are dried without lids at 80° C. for 2 h and, after cooling, are reweighed.
b) Calculation:

$$\begin{matrix}\frac{{m}_{0} Ai - {m}_{1} Ai}{{mT}_{0} {A}_{i}} * 100 = Δ Ai ( \% loss of tablet stored in air ) \\ \frac{{m}_{0} Ac - {m}_{1} Ac}{m {T}_{0} Ac} * 100 = Δ Ac ( \% loss of tablet stored in acetone ) \\ Δ Ac - Δ Ai = \% extractables\end{matrix}$$

mT₀Ai Mass of tablet Ai before storage in air

m₀Ai Mass of beaker+tablet Ai before storage in air

m₁Ai Mass of beaker+tablet Ai after storage in air

mT₀Ac Mass of tablet Ac before storage in acetone

m₀Ac Mass of beaker+tablet Ai before storage in acetone

m₁Ac Mass of beaker+tablet Ai after storage in acetone

c) Blank Sample

The blank sample tested along with each determination (½ tablet 24 h in air) is used to detect any losses of material in the course of drying. From experience, all blank samples lose 0.2%-0.5% on drying. This loss is subtracted from the loss of the extracted sample.

### EXAMPLE 1

### Reaction of TMP with Methyl Acrylate in MTBE

A mixture of 0.1 mol (13.4 g) of trimethylolpropane (TMP), 1.0 mol (86.1 g) of methyl acrylate, 200 ml of MTBE, 20 g of 5 Å mol sieve and 2.0 g of Novozym 435 (lipase from Candida antarctica B) was stirred under reflux for 24 h. The enzyme was removed by filtration, MTBE was taken off on a rotary evaporator under reduced pressure, and 22 g of crude product (a clear, yellowish liquid) were obtained.

A sample was taken, silylated, and analyzed by GC. According to GC analysis the composition of the product was as follows: 16% TMP, 60% TMP monoacrylate, 21% TMP diacrylate, <1% TMP triacrylate.

### EXAMPLE 2

### Reaction of Glycerol with Methyl Acrylate in Acetone, Without Mol Sieve

A mixture of 125 mmol (11.5 g) of glycerol, 1.25 mol (107.6 g) of methyl acrylate, 250 ml of acetone and 2.5 g of Novozym 435 (lipase from Candida antarctica B) was shaken at 40° C. for 2 days. The enzyme was removed by filtration (it can be used again) and acetone was taken off on a rotary evaporator under reduced pressure. This gave 27 g of crude product (a clear, yellowish liquid).

A sample was taken, silylated, and analyzed by GC. According to GC analysis the composition of the product was as follows: 6% glycerol, 54% glycerol monoacrylate, 37% glycerol diacrylate, <1% glycerol triacrylate.

Total extractables after thermal or UV cure: <5% by weight

### EXAMPLE 3

### Reaction of TMP with Methyl Acrylate

a) A mixture of 0.5 mol (67 g) of TMP, 5 mol (430.5 g) of methyl acrylate, 100 g of mol sieve (5 Å) and 10 g of Novozym 435 (lipase from Candida antarctica B) was stirred at 60° C. for 72 hours. The enzyme was removed by filtration and the filtrate was separated from the constituents of low volatility by distillation. This gave 142 g of TMPTA (a clear, colorless liquid).

A sample was taken and silylated. According to GC analysis >99% of the TMP had undergone reaction, i.e., the triacrylate was formed almost completely.

Total extractables after UV cure: <5% by weight

b) A mixture of 0.5 mol (67 g) of TMP, 5 mol (430.5 g) of methyl acrylate, 100 g of mol sieve (5 Å) and 10 g of Novozym 435 (lipase from Candida antarctica B) was stirred at 40° C. for 24 h. The enzyme was removed by filtration and the filtrate was separated from the constituents of low volatility by distillation. This gave 104 g of product (a clear, colorless liquid).

A sample was taken and silylated. According to GC analysis the composition of the product was as follows: 2% TMP, 22% TMP monoacrylate, 72% TMP diacrylate, <3% TMP triacrylate.

Total extractables after thermal or UV cure: <5% by weight

### EXAMPLE 4

### Reaction of TMP with Acrylic Acid (Comparative Example 1)

A mixture of 0.5 mol (67 g) of TMP, 0.5% by weight of H₂SO₄, 1.8 mol (99 g) of acrylic acid was dissolved in cyclohexane and water of reaction obtained was removed up to a conversion of 50% or 66%. The batch was in each case purified by distillation to an acid number of 40. This gave 108 g or 120 g of product (clear, yellowish liquids).

A sample was taken and silylated. According to GC analysis the composition of the product was as follows:

Conversion [50%]: 15% TMP, 45% TMP monoacrylate, 23% TMP diacrylate, 17% TMP triacrylate.

Total extractables after thermal cure: 33% by weight (butyl acetate, room temp.)

Total extractables after UV cure: 47% by weight (butyl acetate, room temp.)

Conversion [67%]: 2% TMP, 15% TMP monoacrylate, 25% TMP diacrylate, 59% TMP triacrylate.

Total extractables after thermal cure: 64% by weight (butyl acetate, room temp.)

Total extractables after UV cure: 27% by weight (butyl acetate, room temp.)

### EXAMPLE 5

### Reaction of Glycerol with Ethyl Acrylate in Tert-Butanol

A mixture of 5 mmol (0.46 g) of glycerol, 50 mmol (5.0 g) of ethyl acrylate, 10 ml of tert-butanol, 1 g of mol sieve (5 Å) and 0.1 g of Novozym 435 (lipase from Candida antarctica B) was shaken at 20° C. for 3 days.

A sample was taken, silylated, and analyzed by GC. According to GC analysis the composition of the product was as follows: 5% by weight glycerol, 42% by weight glycerol monoacrylate, 53% by weight glycerol diacrylate and <1% by weight glycerol triacrylate.

### EXAMPLE 6

### Reaction of Glycerol with Methyl Acrylate

A mixture of 125 mmol (11.5 g) of glycerol, 1.25 mol (107.6 g) of methyl acrylate, 250 ml of acetone and 2.5 g of Novozym 435 (lipase from Candida antarctica B) was shaken at 40° C. for 2 days. The enzyme was removed by filtration (and can be reused). Acetone was removed in a rotary evaporator under reduced pressure. This gave 19.4 g of crude product (a clear, yellowish liquid).

A sample was taken, silylated, and analyzed by GC. According to GC analysis the composition of the product was as follows: 15% by weight glycerol, 37% by weight glycerol monoacrylate, 46% by weight glycerol diacrylate and <1% by weight glycerol triacrylate.

### EXAMPLE 7

### Reaction of Glycerol and Methyl Acrylate in Acetone

A mixture of 0.5 mol (46.3 g) of glycerol, 5 mol (430.5 g) of methyl acrylate, 500 ml of acetone, 100 g of mol sieve (5 Å) and 10.0 g of Novozym 435 (lipase from Candida antarctica B) was stirred at 20° C. for 72 hours. The enzyme was removed by filtration (and can be reused) and the filtrate was concentrated under reduced pressure. This gave 80.9 g of crude product (a clear, colorless liquid).

A sample was taken and silylated. According to GC analysis the composition of the product was as follows: 8% by weight glycerol, 48% by weight glycerol monoacrylate, 41% by weight glycerol diacrylate and 3% by weight glycerol triacrylate.

### EXAMPLE 8

### Reaction of Glycerol and Methyl Methacrylate Without Solvent or Mol Seive

A mixture of 5 mmol (0.46 g) of glycerol, 50 mmol (5.0 g) of methyl methacrylate and 0.1 g of Novozym 435 (lipase from Candida antarctica B) was shaken at 20° C. for 24 hours.

A sample was taken and silylated. According to GC analysis the composition of the product was as follows: 15% by weight glycerol, 55% by weight glycerol monomethacrylate, 30% by weight glycerol dimethacrylate and <1% by weight glycerol trimethacrylate.

### EXAMPLE 9

### Reaction of Erythritol and Methyl Acrylate in Tert-Butanol

50 mmol of erythritol (6.1 g), 500 mmol of methyl acrylate, 300 ml of tert-butanol and 1.0 g of immobilized lipase from Candida antarctica (Novozym 435) were stirred at 40° C. for 72 hours. The enzyme was removed by filtration and the excess methyl acrylate and the solvent were removed on a rotary evaporator under reduced pressure at 40° C.

This gave 14.1 g of target product which according to GC analysis contained 21% by weight erythritol, 49% by weight erythritol monoacrylate, 29% by weight erythritol diacrylate and <0.2% by weight erythritol triacrylate.

### EXAMPLE 10

### Reaction of Sorbitol with Methyl Acrylate in Tert-Butanol

In a four-necked round-bottom flask surmounted with a reflux condenser 63.8 g of sorbitol (0.35 mol), 301.3 g of methyl acrylate (3.5 mol), 2100 ml of tert-butanol and 7.0 g of lyophilized lipase from Burkholderia sp. were stirred at 40° C. for 72 hours. The mixture was then filtered using a suction filter (D3 with silica gel layer) to remove the lipase and undissolved sorbitol, and excess methyl acrylate and solvent were removed on a rotary evaporator under reduced pressure at 40° C. This gave 83.3 g of product.

GC analysis gave a result of 45% by weight sorbitol monoacrylate, 42% by weight sorbitol diacrylate, 3% by weight sorbitol triacrylate and 10% by weight sorbitol.

### EXAMPLE 11

### Preparation of a Cured Varnish Coat

a) Thermal Curing:

A mixture of 16% by weight of a reaction product from example 3b and, respectively, 2, 50% by weight of Basonat HI 100, 34% by weight of a polyol, and a mixture of 3.5% by weight Irgacure® 184 (Ciba Specialty Chemicals) and 0.5% by weight Lucirin TPO® (BASF AG) were dissolved in butyl acetate, with the addition of 1% by weight DBTL, and the solution was subjected to thermal curing at 60° C. for 16 h. This gave a colorless film which after 30 minutes was tack-free. This film was cooled after 16 h, extracted with acetone at RT for 24 h, and then dried.

b) UV Curing:

The coating composition was exposed five times under an undoped high-pressure mercury lamp (output 120 W/cm) with a lamp-to-substrate distance of 12 cm at a belt speed of 5 m/min. The coat thickness after exposure was about 50 μm.

The pendulum damping was determined in accordance with DIN 53157 to be 118 and 110, respectively, and is a measure of the hardness of the coating. The result is stated in pendulum swings. High values in this case denote high hardness. The Erichsen cupping was determined in accordance with DIN 53156 to be 4.6 and 7.0, respectively, and is a measure of the flexibility and elasticity. The result is given in millimeters (mm). High values denote high flexibility. The adhesion with cross-cutting was determined in accordance with DIN 53151 and reported as a rating. Low values denote high adhesion. This resulted in each case in a 0/5 assessment.

For comparative example 1 [50%] the values obtained are as follows:

Pendulum damping: 32; Erichsen cupping: 8.9; adhesion: ⅕.

It is therefore apparent that using the polyol acrylates of the invention it is possible to produce polymer coatings having a markedly improved profile of properties.

1-22. (canceled)

23. A process for the enzymatic synthesis of polyol acrylates, in which an aliphatic polyol is reacted with an acrylic acid compound or an alkyl ester thereof in bulk or in a liquid reaction medium comprising an organic solvent, in the presence of an enzyme which is selected from hydrolases and transfers acrylate groups, and after the end of the reaction the polyol acrylate(s) formed is (are) isolated optionally from the reaction mixture.

24. A process as claimed in claim 23, wherein the liquid reaction medium has an initial water content of less than about 10% by volume.

25. A process as claimed in either of the preceding claims, wherein acrylic acid compound and polyol are used in a molar ratio of about 100:1 to 1:1.

26. A process as claimed in claim 23, wherein the acrylic acid compound is acrylic acid, lower-alkyl-substituted acrylic acid, or the alkyl esters of these compounds, or mixtures thereof.

27. A process as claimed in claim 23, wherein the polyol is a straight-chain or branched or carbocyclic, saturated or unsaturated hydrocarbon compounds having at least 3 carbon atoms or at least 3 (esterifiable) hydroxyl groups in optically pure form or as a stereoisomer mixture, or mixtures of different polyols.

28. A process as claimed in claim 27, wherein the polyol is a straight-chain, branched or cyclic saturated hydrocarbons having 3 to 30 carbon atoms and from 3 to 10 hydroxyl groups.

29. A process as claimed in claim 23, wherein a completely acrylated polyol acrylate is added to the reaction medium, the polyol acrylate being the ester of an acrylic acid compound and a polyol as defined in claim 23.

30. A process as claimed in claim 23, wherein the polyol is glycerol, diglycerol, triglycerol, 1,2,4-butanetriol, trimethylolmethane, trimethylolethane, trimethylolpropane, trimethylolbutane, 2,2,4-trimethyl-1,3-pentanediol, pentaerythritol, ditrimethylolpropane, dipentaerythritol, tripentaerythritol, D-, L-, and mesoerythritol, D- and L-arabitol, adonitol, xylitol, sorbitol, mannitol, dulcitol or inositols, or the mixtures or alkoxylates thereof.

31. A process as claimed in claim 23, wherein the hydrolase is esterases (E.C. 3.1.-.-).

32. A process as claimed in claim 31, wherein the esterase is lipases (E.C. 3.1.1.3), glycosylases (E.C. 3.2.-.-), or proteases (E.C. 3.4.-.-) in free or immobilized form.

33. A process as claimed in claim 23, wherein the organic solvent is C₁-C₆ alkanols, pyridine, polyalkylene glycol dialkyl ethers, alkylene carbonate, C₁-C₆ alkyl alkanecarboxylic esters, acetone, 1,4-dioxane, 1,3-dioxolane, THF, dimethoxymethane, dimethoxyethane, or mixtures thereof.

34. A process as claimed in claim 23, wherein the enzyme content of the reaction medium is in the range from about 0.01 to 10% by weight, based on the polyol used.

35. A process as claimed in claim 23, wherein the reaction temperature is in the range from 0 to about 100° C.

36. A process as claimed in claim 23, wherein the reaction medium is single-phase or multiphase and wherein the reactants are present in solution, suspension or emulsion.

37. A process as claimed in claim 23, wherein alcohol produced during the transesterification or water of reaction produced during the esterification is removed from the reaction equilibrium.

38. A process for preparing polymeric polyol acrylates, wherein at least one polyol acrylate is prepared by a process as claimed in claim 23, optionally separating from the reaction mixture, and optionally polymerizing together with further comonomers.

39. A process as claimed in claim 37, wherein a reaction product comprising substantially polyol monoacrylates is reacted with at least one comonomer to form a linear copolymer.

40. A polymeric polyol acrylate obtainable by a process as claimed in claim 38.

41. A reaction product comprising polyol acrylate, obtainable by a process as claimed in claim 23.

42. A reaction as claimed in claim 41 containing from about 60 to 100 mol %, based on the total molar number of polyol acrylate, of compounds having both alcohol functionalization and acrylate functionalization.

43. A process for the preparation of coating materials which comprises using the polymeric polyol acrylate as claimed in claim 40.

44. The process as claimed in claim 43, wherein the coating materials are thermally curable and have a total extractable fraction after thermal curing, of less than 20% by weight.

45. The process as claimed in claim 44, wherein after thermal curing alone the coatings are not tacky.
